(19) United States
(12) Patent Application Publication
(10) Pub. No.: US 2024/0180207 A1
(43) Pub. Date: Jun. 6, 2024

(54) AFLATOXIN REDUCTION IN NUTS VIA UV AND/OR OXIDATION

(71) Applicant: OLAM AMERICAS,LLC, Fresno, CA (US)

(72) Inventors: Jacob Sebastian, Oak Park, IL (US); John Martin, Oak Park, IL (US)

(21) Appl. No.: 18/526,822

(22) Filed: Dec. 1, 2023

Related U.S. Application Data
(60) Provisional application No. 63/429,739, filed on Dec. 2, 2022.

(51) Int. Cl. A23L 3/28; A23B 7/015; A23B 7/10; A23B 7/152; A23B 7/157; A23B 9/06; A23B 9/22; A23B 9/30; A23L 3/32; A23L 3/3445; A23L 3/3508; A23L 3/358

(52) U.S. Cl. CPC A23L 3/28; A23B 7/015; A23B 7/10; A23B 7/152; A23B 7/157; A23B 9/06; A23B 9/22; A23B 9/30; A23L 3/32; A23L 3/3445; A23L 3/3508; A23L 3/358

(57) ABSTRACT

The disclosure provides a method of reducing aflatoxin contamination in one or more nuts and/or reducing the amount of Aspergillus flavus and/or Aspergillus parasiticus resulting in the presence of aflatoxin contamination in one or more nuts. In an embodiment, nuts are treated with ultraviolet (UV) light, ozone, and peroxide, or combinations thereof. In an embodiment, nuts are treated with UV light and ozone. In an embodiment, nuts are treated with UV light and peroxide. In an embodiment, nuts are treated with ozone and peroxide.

### CROSS-REFERENCE TO RELATED APPLICATIONS

The present application claims the priority benefit of U.S. Provisional Application No. 63/429,739, filed on Dec. 2, 2022, which application is hereby incorporated herein by reference in its entirety.

### FIELD

This disclosure relates to methods of reducing aflatoxin contamination of a foodstuff, such as nuts.

### TECHNICAL FIELD

Aflatoxins are a group of structurally related mycotoxins. Aflatoxins are produced by specific Aspergillus species and can be toxic, mutagenic, and/or carcinogenic. Aspergillus flavus and Aspergillus parasiticus are the main toxigenic species. A. flavus is not host-specific and infects a variety of food crops, while A. parasiticus is more host specific, and in particular, may contaminate peanuts. See e.g., Jallow et al, 2021, Compr Rev Food Sci Food Saf 20: 2332-2381).

Aflatoxin contamination and fungal invasion are found in agricultural products and foods, such as nuts, cocoa, and spices, and consequently pose a large problem for consumer safety. Supply chain detection practices can mitigate consumer exposure but are accompanied by substantial economic losses since contaminated food products are destroyed. The present disclosure is directed to solutions to this problem.

### SUMMARY

This disclosure is generally related to a method of reducing aflatoxin contamination in one or more nuts and/or reducing the amount of Aspergillus flavus and/or Aspergillus parasiticus resulting in aflatoxin contamination on one or more nuts. In aspects of the method, the method comprises contacting one or more nuts with one or more treatments, thereby reducing the presence of aflatoxin and/or reducing the amount of Aspergillus flavus and/or Aspergillus parasiticus resulting in the presence of aflatoxin, relative to the one or more nuts prior to contact with the one or more treatments. In aspects of the method, the method comprises contacting one or more nuts with one or more treatments, thereby reducing the presence of aflatoxin, relative to the one or more nuts prior to contact with the one or more treatments. In aspects of the method, the method also reduces the presence of Aspergillus flavus and/or Aspergillus parasiticus, relative to the one or more nuts prior to contact with the one or more treatments. Treatments include engaging the one or more nuts with an oxidizing agent, such as ultraviolet (UV) light, ozone (O₃), peroxide (e.g., hydrogen peroxide H₂O₂), or electrolyzed oxidizing water (EOW), pulsed electric field (PEF), supercritical carbon dioxide, cold plasma, organic acid wash, and combinations of one or more thereof. For example, a treatment may include exposing one or more nuts to UV light, as well as exposing the one or nuts to one or more oxidizing compounds such ozone or peroxide, e.g. through gaseous exposure and/or exposure in a solvent or solution, such as an aqueous ozone treatment. UV light and oxidizing compounds may be applied concurrently, or sequentially in any order.

### BRIEF DESCRIPTION OF THE DRAWINGS

The accompanying drawings are incorporated in and constitute a part of this specification and illustrate various methods and compositions disclosed herein.

FIGS. 1A, 1B, and 1C depict data for Aspergillus-contaminated hazelnuts (FIG. 1A) and almonds (1B) subjected to ultraviolet (UV) irradiation at a fixed power for different lengths of time (1 hour, 4 hours, and 36 hours). “Native control”—nuts not contaminated with Aspergillus. “After inoculation and incubation”—nuts inoculated with Aspergillus and incubated to obtain Aspergillus-contaminated nuts. FIG. 1C depicts a summary of the final data for both hazelnuts and almonds. See Example 1c.

FIGS. 2A and 2B depict data for Aspergillus-contaminated hazelnuts and almonds subjected to gaseous ozone treatment (hazelnuts) or aqueous ozone treatment (almonds). Two dosages were tested for each ozone treatment. Aspergillus cell counts (FIG. 2A) and aflatoxin (FIG. 2B; sum of aflatoxin B1 and B2) were assessed before and after treatment. A treatment control was also assessed. See Example 2c.

FIG. 3 depicts data related to three different Aspergillus strains tested for growth and aflatoxin production on clean hazel nuts using two sets of incubation conditions and six timepoints. See Example 10.

### DETAILED DESCRIPTION

### Definitions & Abbreviations

Unless defined otherwise, all technical and scientific terms used herein generally have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +/−10%, from the specified value, as such variations are appropriate to perform the disclosed methods.

As used herein, the term “aflatoxin” refers to a chemical substance that is part of a group of structurally related mycotoxins that are produced by certain species of fungi, such as Aspergillus flavus and Aspergillus parasiticus. Aflatoxins can be toxic, mutagenic and/or carcinogenic. Aflatoxins from the B-series (aflatoxins B1 and B2), the G-series (aflatoxins G1 and G2), and M-series are some of the main types of interest and are regulated for safety of food and agricultural products. Aflatoxin B1 (AFB1) is the most potent carcinogen of the aflatoxins.

As used herein, the terms “reduction of aflatoxin contamination”, “reducing aflatoxin contamination”, and “aflatoxin reduction” refers to reducing the amount of aflatoxin, such as AFB1, detectable in a foodstuff, such as a nut, or a plurality of nuts. The terms encompass decontamination, detoxification, and both decontamination and detoxification. Decontamination refers to the physical removal of the aflatoxin and/or removal of Aspergillus flavus and Aspergillus parasiticus. Detoxification refers to the degradation of the aflatoxin, such as AFB1, and/or Aspergillus flavus and Aspergillus parasiticus.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is understood that any and all whole or partial integers between any ranges set forth herein are included herein.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

Finally, the steps of all methods described herein are performable in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

### DESCRIPTION

Embodiments of the present invention are described below. It is, however, expressly noted that the present invention is not limited to these embodiments, but rather the intention is that modifications that are apparent to the person skilled in the art and equivalents thereof are also included.

This disclosure is generally related to a method of reducing aflatoxin contamination in on one or more nuts and/or reducing the amount of Aspergillus flavus and/or Aspergillus parasiticus resulting in the presence aflatoxin contamination in one or more nuts. In aspects of the method, the method comprises contacting one or more nuts with one or more treatments, thereby reducing the amount of aflatoxin and/or reducing the presence of Aspergillus flavus and/or Aspergillus parasiticus resulting in the aflatoxin contamination, relative to the one or more nuts prior to contact with the one or more treatments.

The extent of aflatoxin contamination can vary with the type of nut. Moreover, for a given type of nut, the extent of aflatoxin contamination can depend on the source of the nuts. For instance, a global assessment of aflatoxin contamination of various nuts from countries in Asia, Africa, Europe and South America, found the mean concentration of total aflatoxin (sum of AFB1, AFB2, AFG1 and AFG2) in peanuts was 40.87 ppb, with a range of 0 ppb (not detected) to 530 ppb (Ebrahimi et al, The prevalence of aflatoxins in different nut samples: A global systematic review and probabilistic risk assessment. AIMS Agriculture and Food, 2022, 7(1): 130-148). Similarly, the mean concentration of total aflatoxin in pistachios was 37.52 ppb, with a range of 0 ppb (not detected) to 245.6 ppb. The mean concentration of total aflatoxin in almonds was 3.54 ppb, with a range of 0 ppb (not detected) to 32.9 ppb, and in hazelnuts, the mean concentration of total aflatoxin was 17.33 ppb, with a range of 0.2 ppb to 124 ppb. The permitted aflatoxin contamination on nuts for consumption varies from country to country. The EU has some of the strictest levels. For instance, peanuts intended for direct human consumption or use as an ingredient in foodstuffs can have a maximum of 4 ppb aflatoxins (sum of AFB1, AFB2, AFG1 and AFG2). Almonds, pistachios, hazelnuts and Brazil nuts that are intended for direct human consumption or use as an ingredient in foodstuffs can have a maximum of 10 ppb aflatoxin

In certain aspects, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is subjected to at least one treatment, thereby reducing the amount of aflatoxin to less than 300 ppb and/or reducing the presence of Aspergillus flavus and/or Aspergillus parasiticus to an amount that results in less than 300 ppb aflatoxin. In certain embodiments, the amount of aflatoxin is reduced by at least one log (e.g., reduced to 30 ppb), or at least two logs (e.g., reduced to 3 ppb). In certain aspects, one or more nuts having an amount of aflatoxin of at least about 200 parts-per-billion (ppb) and/or presence of Aspergillus flavus and/or Aspergillus parasiticus in an amount resulting in at least about 200 ppb aflatoxin is subjected to at least one treatment, thereby reducing the amount of aflatoxin to less than 200 ppb and/or reducing the presence of Aspergillus flavus and/or Aspergillus parasiticus to an amount that results in less than 200 ppb aflatoxin. In certain embodiments, the amount of aflatoxin is reduced by at least one log (e.g., reduced to 20 ppb), or at least two logs (e.g., reduced to 2 ppb).

In some embodiments, the methods of the disclosure reduce the amount of aflatoxin by at least about 10%, at least about 20%. at least about 30%, at least about 40%, at least about 50%, at least about 60%, or at least about 70%, at least about 80%, or at least about 90%.

In some embodiments, the methods of the disclosure reduce the amount of aflatoxin by at least about 60%, at least about 65%, at least about 70%, at least about 75%, at least about 80%, at least about 85%, or at least about 90%.

In some embodiments, the methods of the disclosure reduce the amount of aflatoxin by at least about 91%, at least about 92%, at least about 93%, at least about 94%, at least about 95%, at least about 96%, at least about 97%, at least about 98%, or at least about 99%.

In some embodiments, the methods of the disclosure reduce the amount of aflatoxin by at least about 0.5 log, at least about 1 log, at least about 1.5 log, at least about 2 log, or at least about 2.5 log.

In some embodiments, the methods of the disclosure reduce the amount of aflatoxin to less than about 200 ppb, less than about 190 ppb, less than about 180 ppb, less than about 170 ppb, less than about 160 ppb, less than about 150 ppb, less than about 140 ppb, less than about 130 ppb, less than about 120 ppb, less than about 110 ppb, less than about 100 ppb, or less than about 10 ppb.

In some embodiments, the methods of the disclosure reduce the amount of aflatoxin to less than about 95 ppb, less than about 90 ppb, less than about 85 ppb, less than about 80 ppb, less than about 75 ppb, less than about 70 ppb, less than about 65 ppb, less than about 60 ppb, less than about 55 ppb, or less than about 50 ppb.

In some embodiments, the methods of the disclosure reduce the amount of aflatoxin to less than about 45 ppb, less than about 40 ppb, less than about 35 ppb, less than about 30 ppb, less than about 25 ppb, less than about 20 ppb, less than about 15 ppb, less than about 10 ppb, less than about 5 ppb, less than about 4 ppb, or 2 ppb or less.

As used herein, “nut” refers to an edible nut. Exemplary edible nuts include, but are not limited to, almonds, cashews, hazelnuts, macadamias, peanuts, pecans, pistachios, and walnuts. In the methods of the disclosure, nuts for treatment can include their shells (shelled nuts) or can have their shells removed (unshelled nuts). Crushed unshelled nuts, including but not limited to, cracked nuts, sliced nuts, nut butters, and nut pastes, can also be treated in the methods of the disclosure.

Aspects of the present disclosure addresses the issue of aflatoxin contamination with minimal integrity impact of the underlying food product. As an example, when one or more aflatoxin reduction processes are used on nut products, the nuts are not cooked as a side-effect of the mitigation technology, and no residual chemicals are present at a level that would prevent the nut being sold in the same product category or adversely affect user consumption experience.

The method of the disclosure comprises subjecting one or more nuts to one or more treatments, thereby reducing the amount of aflatoxin and/or reducing the presence of Aspergillus flavus and/or Aspergillus parasiticus resulting in the presence of aflatoxin contamination. Exemplary treatments include contacting the one or more nuts with an oxidizing agent such as ultraviolet (UV) light, ozone (O₃), and/or peroxide (e.g., hydrogen peroxide H₂O₂), electrolyzed oxidizing water (EOW), pulsed electric field (PEF), cold plasma, supercritical fluid extraction (SFE) using supercritical carbon dioxide (SC CO₂), one or more chemical washes, and combinations thereof. Other potential oxidizing agents include, for example, hypochlorites like sodium chlorite, or oxidant gases such as oxygen (O₂) or nitrous oxide. In one aspect, the nuts can be subjected to multiple treatments in series or in parallel.

### Ultraviolet (UV) Light

In an embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with ultraviolet (UV) light, thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb). Without being bound by theory, the inventors believe that the high energy photons of UV light have sufficient energy to break bonds within organic molecules, and in the case of aflatoxin, this may lead to hydration of the furan ring—the site on the molecule that leads to aflatoxins' toxicity. Another potential advantage of using UV light includes the generation of additional reactive species, such as oxygen radicals, which a can subsequently react with and degrade the aflatoxin and their primary breakdown products.

Ultraviolet light spectrum spans wavelengths of about 10 nanometers (nm) to about 400 nm. UV light spectrum is considered to have three regions: UV-A, UV-B, and UV-C. UV-A light encompasses wavelengths of about 320 to about 400 nm. UV-B light encompasses wavelengths of about 280 to about 320 nm, and UV-C light encompasses wavelengths of about 100 to about 280 nm. UV light treatment of nuts can comprise any of the three UV regions. UV-C light may oxidize nut oil which may adversely affect the taste. UV light treatment can be monochromatic (substantially single wavelength) or polychromatic (a range of wavelengths). In certain embodiments, the UV light treatment is UV-A light. In some embodiments, the UV-A light treatment is monochromatic, around 350 nm or around 365 nm. In some embodiments, the UV-A light treatment is polychromatic, such as about 315 nm to about 400 nm or about 345 nm to about 400 nm. In some embodiments, the UV light treatment is polychromatic, such as about 140 nm to about 365 nm, about 350 nm to about 365 nm, or about 140 nm to about 350 nm. In certain embodiments, the UV light treatment is UV-C light. In some embodiments, the UV-C light treatment is monochromatic, around 256 nm (e.g. about 256 nm, or about 251-261 nm, or about 255-257 nm), around 254 nm (e.g. about 254 nm, or about 249-259 nm, or about 253-255 nm), or around 142 nm (e.g. about 142 nm, or about 137-147 nm, or about 141-143 nm). In some embodiments, the UV-C light treatment is less than 256 nm, less than 254 nm, less than 225 nm, less than 190 nm, or less than 143 nm. In combination treatments, where the second treatment may be adversely affected by higher wavelengths, for instance, ozone, the UV-C light is less than 190 nm, such as about 186 nm or 142 nm, to minimize possible inactivation of ozone at higher wavelengths. UV treatment can be from about 15 minutes to about 4 hours, or about 15 minutes to about 1 hour, or about 30 minutes to about 1 hour, or about 30 minutes to about 2 hours, such as about, or no more than, 15 min, 30 min, 1 hr, 2 hr, 3 hr, or 4 hr. UV treatment can also be performed for greater than 4 hours, such as but not limited to about 6 hours, about 12 hours, about 24 hours, or longer. However, extended UV treatment may contribute to additional aflatoxin generation during the treatment, which reduces the overall efficacy. In some embodiments, rotation of the target (nuts) during the UV treatment can expose more surface to the treatment which can improve the effect. For instance, multiple passes on multihead conveyors can be used to rotate nuts during exposure to the UV treatment. UV light treatment can also be delivered in water, which can provide an additional source of reactive species.

### Example 1a

Peanuts are obtained and the aflatoxin content is measured, for instance by HPLC, and is determined to be on average at least about 300 parts-per-billion (ppb). The peanuts are unshelled. One (1) kilogram of the unshelled peanuts is treated with about 2.75 mW/cm² UV-A light for 30 minutes or 60 minutes. The UV-A light is polychromatic (345 to 400 nm) with a peak output at 365 nm. The aflatoxin content of the UV-A treated peanuts is measured, for instance by HPLC, and determined to be reduced by at least one log from the 300 parts-per-billion (ppb) (e.g., reduced to about 30 ppb or less) for both the 30 min treatment and the 60 min treatment. Treatment conditions are adjusted, resulting in about a two log reduction of aflatoxin.

### Example 1b

Hazelnuts are obtained and the aflatoxin content is measured, for instance by HPLC, and is determined to be on average at least about 200 parts-per-billion (ppb). The hazelnuts are unshelled. One (1) kilogram of the unshelled hazelnuts is treated with about 2.75 mW/cm² UV-A light for 30 minutes or 60 minutes. The UV-A light is polychromatic (345 to 400 nm) with a peak output at 365 nm. The aflatoxin content of the UV-A treated hazelnuts is measured, for instance by HPLC, and determined to be reduced by at least one log from 200 parts-per-billion (ppb) (e.g., reduced to about 20 ppb or less) for both the 30 min treatment and the 60 min treatment.

### Example 1c

This example examined the effect of ultraviolet (UV) irradiation on two types of nuts—hazelnuts and almond. The nuts were subject to Aspergillus (NRRL Catalog No. NRRL 500) inoculation followed by incubation to prepare aflatoxin-contaminated test nuts. The experiment included negative controls (uncontaminated nuts) and positive controls (contaminated nuts subjected to mitigation protocol as much as possible but without the active step—UV irradiation. The nuts were tested in triplicate. As a large a sample of nuts as possible (>30 grams) was used for homogenization of aflatoxin measurements. Samples were then spotted in triplicate on total aflatoxin ELISA plates to maximize reading reliability.

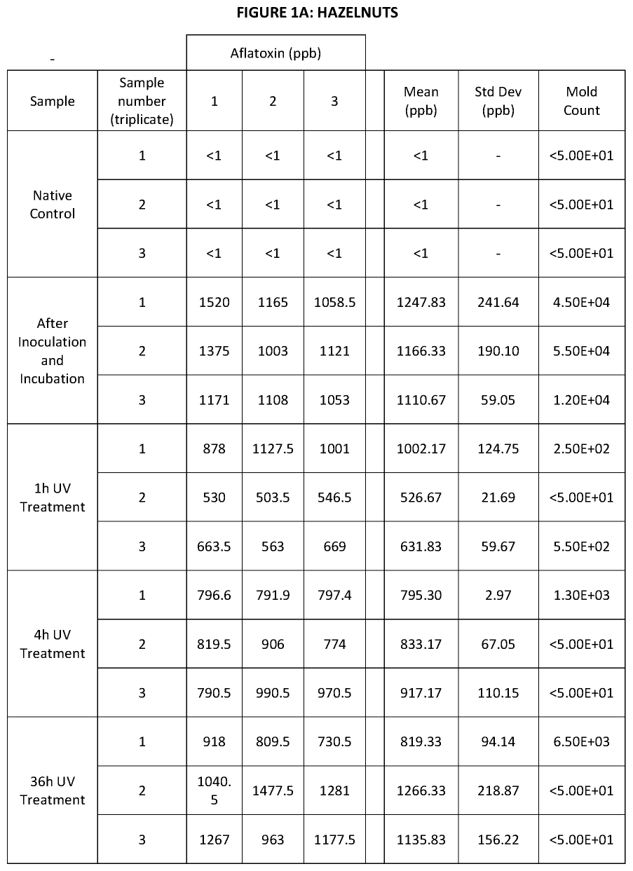

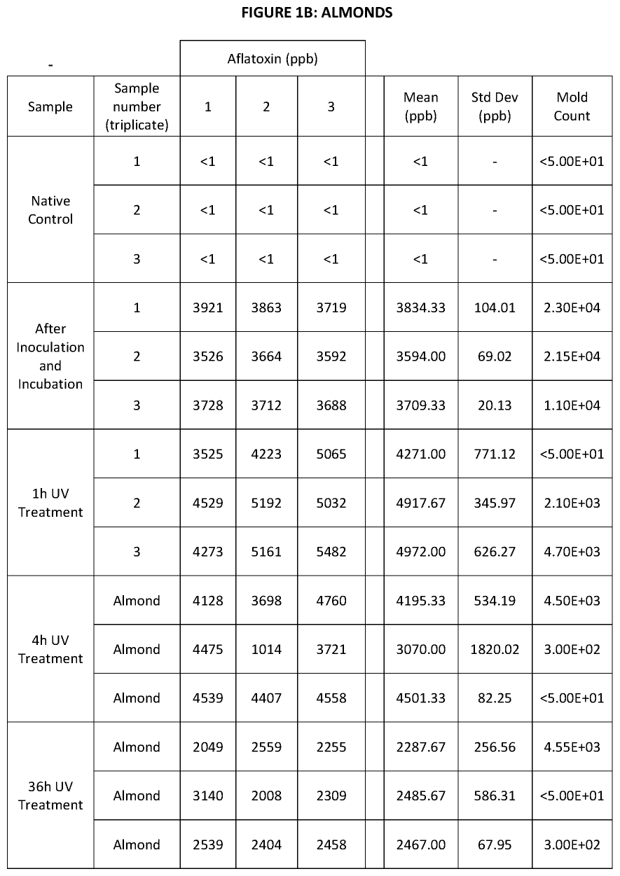

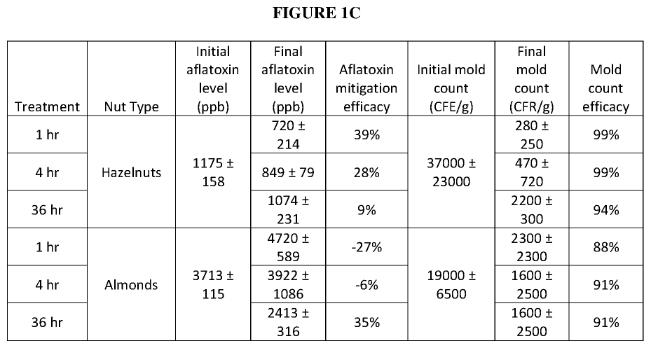

The ultraviolet light treatment had fixed power of 256 nm. Therefore, the parameter examined was time of exposure. Treatments times tested were: 1 hour, 4 hours, and 36 hours. Nuts were mixed four times during a given exposure to improve exposure uniformity. The data for individual replicates are shown in Figure TA (hazelnuts) and FIG. 1B (almonds). The final data for both hazelnuts and almonds are shown FIG. 1C. The treatment was more effective for hazelnuts than for almonds, but only minimally. There was no statistically significant reduction in aflatoxin levels. However, the results contain significant noise. It is believed that additional aflatoxin generation during and after the treatment may be the source of the noise.

### Ozone

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with ozone (O₃), thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

Ozone is a powerful oxidizing agent. Ozone treatment can be in a gaseous form or an aqueous form (ozonized water). Advantageously, gaseous ozone has a multi-hour half life. Aqueous ozone contains additional species that can target aflatoxins and can also provide a washing quality. In certain embodiments, the ozone treatment is gaseous ozone.

Ozone dosage can range from about 3 milligrams per liter (mg/l) to about 300 mg/l, from about 3 mg/l to about 300 mg/l, from about 3 mg/l to about 250 mg/l, from about 3 mg/l to about 240 mg/l, from about 3 mg/l to about 200 mg/l, from about 3 mg/l to about 150 mg/l, from about 3 mg/l to about 125 mg/l, from about 3 mg/l to about 120 mg/l, from about 3 mg/l to about 100 mg/l, from about 3 mg/l to about 50 mg/l, from about 3 mg/l to about 40 mg/l, from about 3 mg/l to about 20 mg/l, from about 3 mg/l to about 15 mg/l, from about 3 mg/l to about 10 mg/l, from about 3 mg/l to about 7.5 mg/l. or from about 3 mg/l to about 6 mg/l.

Duration of ozone treatment can be from about 10 minutes to about 4 days, from about 10 minutes to about 3 days, from about 10 minutes to about 2 days, from about 10 minutes to about 24 hours, from about 10 minutes to about 12 hours, from about 10 minutes to about 6 hours, from about 10 minutes to about 120 minutes, from about 10 minutes to about 60 minutes, from about 10 minutes to about 50 minutes, from about 10 minutes to about 40 minutes, from about 10 minutes to about 30 minutes, from about 10 minutes to about 20 minutes or from about 10 minutes to about 15 minutes. Ozone treatment can be administered at a temperature of about 18° C. to about 30° C., such as about 20° C., about 22° C., about 25° C., about 27° C., about 30° C., about 20° C. to about 22° C., about 20° C. to about 25° C., and about 22° C. to about 27° C. In certain embodiments, the nuts to be treated with ozone have a moisture content of about 3% to about 8%.

In certain embodiments, ozone treatment is gaseous ozone. Gaseous ozone dosage can be from about 120 g/m³ (grams per meter cubed=mg/l) to 250 g/m³, from about 160 g/m³ to 240 g/m³, about 160 g/m³, or about 240 g/m³. In an embodiment, treatment is gaseous ozone at about 160 g/m³ for 120 minutes. In an embodiment, treatment is gaseous ozone at about 240 g/m³ for 60 minutes.

In certain embodiments, ozone treatment is aqueous ozone. Aqueous ozone dosage can be from about 10 g/m³ (grams per meter cubed=mg/l) to 50 g/m³, from about 20 g/m³ to 40 g/m³, about 20 g/m³, or about 40 g/m³. In an embodiment, treatment is aqueous ozone at about 20 g/m³ for 20 minutes. In an embodiment, treatment is aqueous ozone at about 40 g/m³ for 15 minutes. In certain embodiments, ozone treatment may comprise both gaseous ozone and ozone delivered via a liquid medium, e.g. nuts may be exposed to gaseous ozone and then aqueous ozone treatments, or vice versa.

### Example 2a

Hazelnuts are obtained and the aflatoxin content is measured, for instance by HPLC, and is determined to be on average at least about 300 parts-per-billion (ppb). The moisture content of the hazelnuts is measured and determined to be about 3% to about 8%. The hazelnuts are unshelled. One (1) kilogram of the unshelled hazelnuts is treated with 6 mg/l gaseous ozone for about 30 minutes. The aflatoxin content of the ozone treated hazelnuts is measured, for instance by HPLC, and determined to be reduced by at least one log from 300 parts-per-billion (ppb) (e.g., to about 30 ppb or less).

### Example 2b

Pecans are obtained and the aflatoxin content is measured, for instance by HPLC, and is determined to be on average at least about 200 parts-per-billion (ppb). The moisture content of the pecans is measured and determined to be about 3% to about 8%. The pecans are unshelled. One (1) kilogram of the unshelled pecans is treated with 6 mg/l gaseous ozone for about 30 minutes. The aflatoxin content of the ozone treated pecans is measured, for instance by HPLC, and determined to be reduced by at least one log from 200 parts-per-billion (ppb) (e.g., to about 20 ppb or less).

### Example 2c

This example examined the effect of ozone on two types of nuts—hazelnuts and almond. Microbial growth on nuts requires water activity. Therefore, prior to inoculation, the water activity of the test nuts was measured. If the water activity was too low, the test nuts were subject to a humid environment at 30° C. to assure the water activity was at least 0.6. The nuts were subject to Aspergillus (IMI 124931) inoculation followed by incubation to prepare aflatoxin-contaminated test nuts. Three test batches were produced for each type of nut. Treatments were administered at ambient temperature (˜22° C.). Gaseous ozone was applied as high concentration, low temperature (cold) gaseous ozone. Hazelnuts were administered gaseous ozone at an ozone dosage of 240 g/m³ for 60 minutes or an ozone dosage of 160 g/m³ for 120 minutes. Almonds were administered aqueous ozone at an ozone dosage of 20 g/m³ for 20 minutes or an ozone dosage of 40 g/m³ for 15 minutes. Weight gain during the aqueous treatment was monitored; an average weight gain of 9.5% was used for calculation of correct results in the assessment of treatment efficacy.

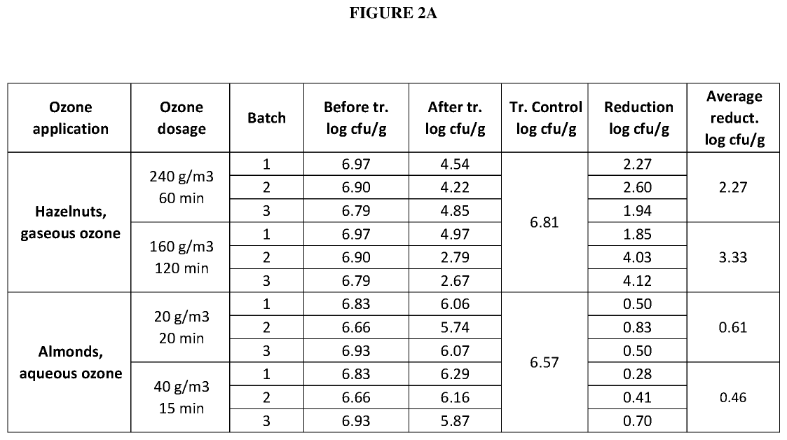

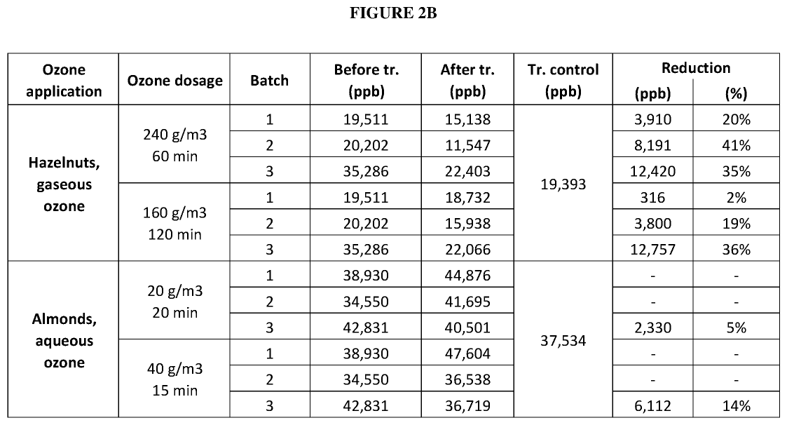

The Aspergillus cell count was measure to assess ozone treatment efficacy in reduction of Aspergillus. The data are shown in FIG. 2A. The data include cell counts of Aspergillus before and after ozone treatment (tr) and the achieved log reductions. The results are averages of triplicate analyses for each batch. For aqueous treatment, the results are corrected for weight gain so that the data can be directly compared to the values before treatment. Both gaseous ozone and aqueous ozone resulting in reduction of Aspergillus. For both gaseous ozone and aqueous ozone, the reduction was greater for the higher concentration ozone for short time condition. Under the conditions tested, the gaseous ozone was more efficacious in reducing Aspergillus cell counts.

The cell count for other mold (other flora) was also measured in these experiments. These data are provided in the Table 1 below and demonstrate that the treatment is also efficacious in reducing counts of other molds.

TABLE 1
Other mold data for ozone treatment

| Ozone | Ozone | | | Before tr. | After tr. | Tr. control |
|---|---|---|---|---|---|---|
| application | dosage | | Batch | Log cfu/g | Log cfu/g | Log cfu/g |
| Hazelnuts | 240 | g/m³ | 1 | 5.67 | 3.80 | 5.96 |
| gaseous | 60 | min | 2 | 6.10 | 2.23 | |
| ozone | | | 3 | 6.15 | 3.32 | |
| | 160 | g/m³ | 1 | 5.67 | 2.07 | |
| | 120 | min | 2 | 6.10 | 0.82 | |
| | | | 3 | 6.15 | 3.27 | |
| Almonds | 20 | g/m³ | 1 | 6.18 | | |
| aqueous | 20 | min | 2 | 6.28 | | |
| ozone | | | 3 | 6.15 | | |
| | 40 | g/m³ | 1 | 6.18 | | |
| | 15 | min | 2 | 6.28 | | |
| | | | 3 | 6.15 | | |

The data for aflatoxin mitigation are shown in FIG. 2B. Aflatoxin B1 and B2 quantities were measured before and after ozone treatment, using milled 100 g samples. For aqueous treatment, the results are corrected for weight gain so that the data can be directly compared to the values before treatment. Both aqueous ozone treatments resulted in reduction of aflatoxin but only for one of the three nut batches tested. Gaseous ozone treatment was efficacious in reducing aflatoxin at both dosages and for all three batches of nuts.

### Peroxide

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with peroxide, thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

In an exemplary method of the disclosure, peroxide concentration can range from about 0.1% to about 30% hydrogen peroxide H₂O₂, about 0.5% to about 25% H₂O₂, about 1% to about 20% H₂O₂, about 10% to about 25% H₂O₂, and values and/or subranges in between, such as about 1%, about 5%, about 10%, about 20% and about 30%. The treatment can be carried out at about 20° C. to about 50° C. for about 1 hour (hr) to about 24 hours, such as about or no more than 0.5 hr, 1 hr, 2 hr, 4, hr, 8 hr or 12 hr. For short duration treatments, the concentration of H₂O₂ should be at the higher end. Lower concentration of H₂O₂ treatment can be efficacious at higher temperature and/or longer duration of contact.

Optionally, peroxidase and/or catalase, which can inactivate H₂O₂, can be inactivated prior to treatment, for instance by a short heat treatment of the nuts for instance about 140° C. for about 10 minutes. Such treatment can yield efficacious H₂O₂ treatment at lower concentrations of H₂O₂ treatment and/or shorter durations of treatment.

### Example 3a

Hazelnuts are obtained and the aflatoxin content is measured, for instance by HPLC, and is determined to be on average at least about 200 parts-per-billion (ppb). The hazelnuts are unshelled. One (1) kilogram of the unshelled hazelnuts is treated with 30% (30 g/gh) hydrogen peroxide (H₂O₂) (at about 2 ml H₂O₂ per gram hazelnuts) at 50° C. for 4 hours. The aflatoxin content of the H₂O₂ treated hazelnuts is measured, for instance by HPLC, and determined to be reduced by at least a half log from the 200 parts-per-billion (ppb) (e.g., reduced to about 63 ppb or less).

### Example 3b

Almonds are obtained and the aflatoxin content is measured, for instance by HPLC, and is determined to be on average at least about 100 parts-per-billion (ppb). The almonds are unshelled. One (1) kilogram of the unshelled almonds is treated with 30% (30 g/gh) hydrogen peroxide (H₂O₂) (at about 2 ml H₂O₂ per gram almonds) at 50° C. for 4 hours. The aflatoxin content of the H₂O₂ treated almonds is measured, for instance by HPLC, and determined be reduced at least one log from the 100 parts-per-billion (ppb) (e.g., reduced to about 10 ppb or less).

### Ozone and Peroxide

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with ozone (O₃) and with peroxide (e.g., H₂O₂), thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

In certain embodiments of the combination treatment, peroxide concentration can range from about 0.1% to about 30% hydrogen peroxide H₂O₂, about 0.5% to about 25% H₂O₂, about 1% to about 20% H₂O₂, about 10% to about 25% H₂O₂, and any values and/or subranges in between, such as about 1%, about 5%, about 10%, about 20% and about 30%. The treatment can be carried out at about 20° C. to about 50° C. The treatment can be carried out for about 1 hour (hr) to about 24 hours, such as about or no more than 0.5 hr, 1 hr, 2 hr, 4, hr, 8 hr or 12 hr. For short duration treatments, the concentration of H₂O₂ should be at the higher end. Lower concentration of H₂O₂ treatment can be efficacious at higher temperature and/or longer duration of contact.

Ozone dosage can range from about 3 milligrams per liter (mg/l) to about 300 mg/l, from about 3 mg/l to about 300 mg/l, and any values and/or subranges in between. Duration of ozone treatment can be from about 10 minutes to about 4 days, and any values and/or subranges in between. Ozone treatment can be administered at a temperature of about 18° C. to about 30° C., and any values and/or subranges in between. Ozone may be gaseous or aqueous. A treatment may comprise treatment with both gaseous and aqueous ozone. Ozone treatment may comprise both gaseous ozone and ozone delivered via a liquid medium, e.g. nuts may be exposed to gaseous ozone and then aqueous ozone treatments, or vice versa.

In certain embodiments, ozone treatment is gaseous ozone. Gaseous ozone dosage can be from about 120 g/m³ (grams per meter cubed=mg/l) to 250 g/m³, from about 160 g/m³ to 240 g/m³, about 160 g/m³, or about 240 g/m³. In an embodiment, treatment is gaseous ozone at about 160 g/m³ for 120 minutes. In an embodiment, treatment is gaseous ozone at about 240 g/m³ for 60 minutes.

In certain embodiments, ozone treatment is aqueous ozone. Aqueous ozone dosage can be from about 10 g/m³ (grams per meter cubed=mg/l) to 50 g/m³, from about 20 g/m³ to 40 g/m³, about 20 g/m³, or about 40 g/m³. In an embodiment, treatment is aqueous ozone at about 20 g/m³ for 20 minutes. In an embodiment, treatment is aqueous ozone at about 40 g/m³ for 15 minutes.

In certain embodiments, ozone treatment may comprise both gaseous ozone and ozone delivered via a liquid medium, e.g. nuts may be exposed to gaseous ozone and then aqueous ozone treatments, or vice versa. Peroxide treatment may precede the ozone treatment, partially or substantially overlap the ozone treatment, or may follow the ozone treatment. In certain embodiments, an aqueous ozone treatment may be followed by a peroxide treatment followed by a gaseous ozone treatment. The peroxide treatment may partially or substantially overlap the aqueous ozone treatment.

### Example 4a

Hazelnuts are obtained and the aflatoxin content is measured, for instance by HPLC, and is determined to be on average at least about 300 parts-per-billion (ppb). The moisture content of the hazelnuts is measured and determined to be about 3% to about 8%. The hazelnuts are unshelled. One (1) kilogram of the unshelled hazelnuts is treated with about 1%, about 10%, or about 30% H₂O₂ at room temperature for 1 hour, followed by treatment with 240 g/m³ gaseous ozone for 10 minutes, 30 minutes or 60 minutes, resulting in nine different combination treatments. The aflatoxin content of the ozone- and peroxide-treated hazelnuts is measured, for instance by HPLC, and determined to be reduced by at least one log from 300 parts-per-billion (ppb) (e.g., to about 30 ppb or less).

### Electrolyzed Oxidizing Water (EOW)

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with electrolyzed oxidizing water (EOW), thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

Electrolyzed oxidizing water (EOW) is produced by electrodialysis of an ionic solution using direct current in an electrolytic chamber, where the anode and cathode are separated by a membrane.

EOW can be acidic electrolyzed oxidizing water (AEOW) which is produced at the anode, neutralized electrolyzed oxidizing which water (NEOW), or basic electrolyzed oxidizing water (BEOW) which is produced at the cathode. NEOW can be produced by adding hydroxide ions to AEOW or can be produced using a single-cell chamber. AEOW can be pH 2.5 to 3.5, oxidation-reduction potential (ORP) 1000-1200 mV, and available chlorine content (ACC) of 30 to 90 ppm. NEOW can be about pH 5.0 to 7, oxidation-reduction potential (ORP) about 700-900 mV, and ACC of 30 to 90 ppm. BEOW can be pH 10 to 13, oxidation-reduction potential (ORP) −795 to −90 OmV, and available chlorine content (ACC) of 80 to 100 ppm.

AEOW and NEOW are preferred in the practice of the method of the disclosure. EOW treatment of nuts can be carried out at a temperature of about −20° C. to about 50° C., about 25° C. to about 45° C., or about 22° C. to about 27° C., or about 25° C. The ratio of volume (ml) of EOW to mass (g) of nuts can be at least about 2:1 (v/m), at least about 3:1 (v/m), at least about 4:1 (v/m), or at least about 5:1 (v/m). Duration of treatment can be from about 5 minutes to about 60 minutes, about 10 minutes to about 30 minutes, about 10 minutes to about 25 minutes, about 10 minutes to about 20 minutes, or about 15 minutes. Treatment can be under static conditions (e.g., nuts not moving around in the EOW) or can be under moving conditions (e.g., nuts moving around in the EOW, such as by oscillation). Moving the nuts, for instance by use of meshed conveyors or multilayered conveyors, during treatment can accelerate reduction of aflatoxin, related to treatment under static conditions, in particular for the about the first 15 minutes of treatment.

### Example 5a

Pistachios are obtained and the aflatoxin content is measured, for instance by HPLC, and is determined to be on average at least about 250 parts-per-billion (ppb). The hazelnuts are unshelled. One (1) kilogram of the unshelled pistachios is treated with AEOW. The AEOW has pH about 2.3, ORP about 1100, and ACC about 70. Treatment is conducted at a ratio of 4:1 (v/m) for 15 minutes at a temperature of about 25° C. under static conditions. The aflatoxin content of the AEOW treated pistachios is measured, for instance by HPLC, and determined to be less reduced by at least one log from 250 parts-per-billion (ppb) (e.g., reduced to about 25 ppb or less).

### Example 5b

Hazelnuts are obtained and the aflatoxin content is measured, for instance by HPLC, and is determined to be on average at least about 200 parts-per-billion (ppb). The hazelnuts are unshelled. One (1) kilogram of the unshelled hazelnuts is treated with AEOW. The AEOW has pH about 2.3, ORP about 1100, and ACC about 70. Treatment is conducted at a ratio of 4:1 (v/m) for 15 minutes at a temperature of about 25° C. under static conditions. The aflatoxin content of the AEOW treated hazelnuts is measured, for instance by HPLC, and determined to be reduced by at about one log from 200 parts-per-billion (ppb) (e.g., reduced to about 20 ppb or less).

### Pulsed Electric Field (PEF)

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with a pulsed electric field (PEF), thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

PEF generates forces within the nuts strong enough to generate and disrupt the pores in living cells, such as Aspergillus fungal cells. PEF can also cause decomposition of aflatoxin. Short electrical impulses (from microseconds to milliseconds each) of high voltage (typically 10-100 kV/cm) are supplied to the product located between the electrodes in the chamber. The process conditions such as electric field strength (kV/cm), pulse frequency, pulse width, shape of the pulse wave and exposure time (related to the flow rate and volume of fluid in the electrode chamber) can be modified as needed to achieve the reduction of aflatoxin. Product can be passed through the electrodes once or more than once to obtain reduction of aflatoxin. PEF treatment parameters are affected by size, geometry and moisture content of the nuts. The energy of the electric pulses used in PEF generates heat. In some cases, it can be useful to include cooling in the process to keep the product being treated at a suitably low temperature. The PEF treatment chamber can be divided into batch treatment chambers for solid material processing, such as batches of nuts.

### Example 6a

Peanuts are obtained and the aflatoxin content is measured, for instance by HPLC, and is determined to be on average at least about 300 parts-per-billion (ppb). The peanuts are shelled. A pilot-scale PEF system is used. The treatment chamber contains two electrodes arranged to enable free fall of the nuts from top to bottom of the chamber between the parallel electrodes. The distance between the electrodes is adjusted to permit nuts to pass through the treatment chamber without any occlusion. The PEF system can apply a maximum 20 kV electric field strength and provides monopolar rectangular pulses. The PEF system is designed to permit nuts to be passed through the field once or more than once. A 10 kilovolt (kV) maximum peak voltages with a frequency of 100 Hertz (Hz), 140 Hz, 160 Hz, or 180 Hz is applied. Unshelled peanuts are subjected to PEF for 1 to 8 cycles. This PEF treatment is described by the level of energy (Joule) because the treatment time and frequency are used to calculate applied energy. PEF treatments range from 0.97 Joule (J) (1 cycle through treatment chamber at 100 Hz) to 17.28 J (8 cycles through treatment chamber at 180 Hz. The aflatoxin content of the PEF treated peanuts is measured, for instance by HPLC, and determined to be reduced by at least one from 300 parts-per-billion (ppb) (e.g., reduced to about 30 ppb or less). Treatment conditions are adjusted, resulting in about at least 1.5 log reduction of aflatoxin (e.g., reduced to about 9.5 ppb or less).

### Example 6b

Hazelnuts are obtained and the aflatoxin content is measured, for instance by HPLC, and is determined to be on average at least about 200 parts-per-billion (ppb). The hazelnuts are shelled. The pilot-scale PEF system described in Example 5a is used. A 10 kilovolt (kV) maximum peak voltages with a frequency of 100 Hertz (Hz), 140 Hz, 160 Hz, or 180 Hz is applied. Unshelled hazelnuts are subjected to PEF for 1 to 8 cycles. This PEF treatment is described by the level of energy (Joule) because the treatment time and frequency are used to calculate applied energy. PEF treatments range from 0.97 Joule (J) (1 cycle through treatment chamber at 100 Hz) to 17.28 J (8 cycles through treatment chamber at 180 Hz. The aflatoxin content of the PEF treated hazelnuts is measured, for instance by HPLC, and determined to be reduced by at least about one from 200 parts-per-billion (ppb) (e.g., reduced to about 20 ppb or less).

### Supercritical Carbon Dioxide

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with supercritical carbon dioxide (SC CO₂), thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

Under high pressure, carbon dioxide (CO₂) enters a supercritical state, wherein the CO₂ acts as both a fluid and a gas. In the supercritical state, CO₂ can act as a selective solvent, where changes in pressure and temperature enable dissolving of specific target compounds. Optionally, for extraction of polar molecules, an additional polar solvent such as ethanol is included in the CO₂, as a polarity modifier to improve extraction of a polar target compound.

In the practice of the method of treatment using supercritical fluid extraction (SFE) using supercritical carbon dioxide (SC CO₂), the pressure can be about 2,000 pounds per square inch (psi) to about 15,000 psi, about 2000 psi to about 10,000 psi, about 2000 psi to about 6000 psi, or about 5000 psi. The temperature can be about 40° C. to about 80° C. about 45° C. to about 60° C., or about 50° C. Duration of the treatment can be about 5 minutes to about 60 minutes, about 5 minutes to about 30 minutes, about 5 minutes to about 15 minutes, such as 5 min, 10 min, 15 min, 30 min, 45 min or 60 min. The SC CO₂ has a polar co-solvent. Exemplary polar co-solvents are ethanol, isopropyl alcohol, or water. In certain embodiments, the polar co-solvent can be present from about 5 to about 20%. In certain embodiments, the ratio of the volume (ml) of polar solvent to the weight (g) of nuts can range from 1 to 4, such as 1.5, 2, 3, or 4.

### Example 7a

Cashews are obtained and the aflatoxin content is measured, for instance by HPLC, and is determined to be on average at least about 300 parts-per-billion (ppb). The cashews are shelled. One (1) kilogram of the unshelled cashews and 10% ethanol (ethanol to nuts ratio of 3 ml/g) are deposited in the extraction chamber, and pressurized with CO₂ to 5000 psi. The treatment is carried out for 15 minutes at 50° C. The aflatoxin content of the SC CO₂ treated cashews is measured, for instance by HPLC, and determined to be reduced by at least one log from the 300 parts-per-billion (ppb), (e.g., to about 30 ppb or less).

### Example 7b

Hazelnuts are obtained and the aflatoxin content is measured, for instance by HPLC, and is determined to be on average at least about 200 parts-per-billion (ppb). The hazelnuts are shelled. One (1) kilogram of the unshelled hazelnuts and 10% ethanol (ethanol to nuts ratio of 3 ml/g) are deposited in the extraction chamber, and pressurized with CO₂ to 5000 psi. The treatment is carried out for 15 minutes at 50° C. The aflatoxin content of the SC CO₂ treated hazelnuts is measured, for instance by HPLC, and determined to be reduced by at least one log from 200 parts-per-billion (ppb) (e.g., to about 20 ppb or less).

### Cold Plasma

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with cold plasma, thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

Exemplary systems, but not limiting, for generating cold atmospheric pressure plasma (CAP or CAPP) include dielectric barrier discharge (DBD) plasma systems and air surface barrier discharge (SBD) systems in practicing the method of the disclosure. Other systems, such as high voltage atmospheric cold plasma system, atmospheric pressure fluidized bed plasma system, and low-pressure cold plasma system, can also be used. Low pressure, high humidity plasma is another exemplary plasma. Exemplary gas includes nitrogen (N₂) and nitrogen-containing mixtures such as N₂ with 0.1% to 1% O₂, for instance N₂+0.1% O₂, and N₂+1% O₂, and ambient air. Optionally, relative humidity (RH) of the gas is between 5% and 80%, such as 20% RH, 40% RH and 80% RH. For an SBD system in the practice of the method, power can be from about 0.18 W/cm to 0.31 W/cm, with an exposure duration of about 1 minutes to about 8 minutes, such as 1 min, 2 min, 4 min or 8 min. For a DBD system in the practice of the method, power can be from about 400 W to about 1150 W, with an exposure duration of about 2 minutes to about 12 minutes, such as 2 min, 4 min, 8 min, 10 min, or 12 min. The exposure duration is inversely related to the power. Higher power can cause an increase in temperature which may not be desirable. Temperature increase can be modulated, for instance, by using a lower power for a longer exposure duration.

### Example 8a

Walnuts are obtained and the aflatoxin content is measured, for instance by HPLC, and is determined to be on average at least about 250 parts-per-billion (ppb). The moisture content of the walnuts is measured and determined to be about 3% to about 8%. The walnuts are unshelled. One (1) kilogram of the unshelled walnuts is treated with cold atmospheric pressure plasma from a DBD system with using N₂+0.1% O₂ at 700 W for 4 minutes. The aflatoxin content of the cold atmospheric pressure plasma-treated walnuts is measured, for instance by HPLC, and determined to be reduced by at least one log from the 250 parts-per-billion (ppb) (e.g., reduced to about 25 ppb or less).

### Example 8b

Hazelnuts are obtained and the aflatoxin content is measured, for instance by HPLC, and is determined to be on average at least about 200 parts-per-billion (ppb). The moisture content of the hazelnuts is measured and determined to be about 3% to about 8%. The hazelnuts are unshelled. One (1) kilogram of the unshelled hazelnuts is treated with cold atmospheric pressure plasma from a DBD system with using N₂+0.1% O₂ at 700 W for 4 minutes. The aflatoxin content of the cold atmospheric pressure plasma-treated hazelnuts is measured, for instance by HPLC, and determined to be reduced by at least one log from the 200 parts-per-billion (ppb) (e.g., reduced to about 20 ppb or less).

### Organic Acid Wash

In another embodiment of the disclosed method, one or more nuts having an amount of aflatoxin of at least about 300 parts-per-billion (ppb) and/or presence of Aspergillus flavus and/or Aspergillus parasiticus in an amount resulting in at least about 300 ppb aflatoxin is contacted with an organic acid solution, thereby reducing the amount of aflatoxin to less than 300 ppb and/or reducing the presence of Aspergillus flavus and/or Aspergillus parasiticus to an amount that results in less than about 300 ppb aflatoxin. In certain embodiments, the amount of aflatoxin is reduced by at least a half log (e.g., reduced to about 95 ppb), by at least one log (e.g., reduced to about 30 ppb), by at least 1.5 log (e.g., reduced to about 9.5 ppb), or at least two logs (e.g., reduced to about 3 ppb).

Organic acids are organic molecules with an acid group. They are generally weak acids, making them less harsh than strong acids, such as hydrochloric acid and sulfuric acid. Exemplary organic acids for practice in the method of the disclosure include citric acid, lactic acid, propionic acid, acetic acid and tartaric acid. In certain embodiments, the organic acid is citric acid, lactic acid or propionic acid. Concentration of the organic acid solution for treatment can be from 1% (w/w) to 10% (w/w), such as 1%, 3%, 5%, 7% or 9%. In certain embodiments, the organic acid solution is from 3% to 7%. Moisture of nuts can impact the effect of chemical wash. Nuts with a higher moisture, such as 16%, can benefit from a higher concentration of organic acid, relative to nuts with a lower moisture, such as 10%. Treatment can be conducted at room temperature, such as about 20° C. to about 30° C., such as 20° C., 22° C., 25° C. or 27° C. Treatment can be conducted for 5 minutes to 30 minutes, such as, 5 minutes, 10 minutes, 15 minutes, 20 minutes, 25 minutes, or 30 minutes. The ratio of nuts to organic acid solution (milliliter) to nuts (grams) can be 0.5 ml/g to 5 ml/g, such as 0.5 ml/g, 1 ml/g, 2.5 ml/g or 5 ml/g. In certain embodiments, the ratio of nuts to organic acid solution (ml) to nuts (grams) can be 1 ml/gram. Treatment can be under static conditions (e.g., nuts not moving around in the organic acid solution) or can be under moving conditions (e.g., nuts moving around in the organic acid solution, such as by oscillation).

### Example 9a

Peanuts are obtained and the aflatoxin content is measured, for instance by HPLC, and is determined to be on average at least about 300 parts-per-billion (ppb). The moisture content of the peanuts is measured and determined to be about 10%. The peanuts are unshelled. One (1) kilogram of the unshelled peanuts is treated with 3% citric acid (at about 1 ml citric acid solution per gram peanuts) at 27° C. for 15 minutes. The aflatoxin content of the organic-acid treated peanuts is measured, for instance by HPLC, and determined to be reduced by at least one log from 300 parts-per-billion (ppb) (e.g., to about 30 ppb or less). Treatment conditions, e.g., concentration, duration of treatment, temperature) are adjusted, resulting in about a two-log reduction of aflatoxin.

### Example 9b

Hazelnuts are obtained and the aflatoxin content is measured, for instance by HPLC, and is determined to be on average at least about 200 parts-per-billion (ppb). The moisture content of the hazelnuts is measured and determined to be about 10%. The hazelnuts are unshelled. One (1) kilogram of the unshelled hazelnuts is treated with 3% citric acid (at about 1 ml citric acid solution per gram hazelnuts) at 27° C. for 15 minutes. The aflatoxin content of the organic-acid treated hazelnuts is measured, for instance by HPLC, and determined be less than 200 parts-per-billion (ppb).

### Combination Treatments

In another aspect, nuts can be subjected to two different technologies, or more than two different technologies. The nuts can be treated with two different technologies at substantially the same time (i.e., in parallel), or can be treated with two different treatments in series, or the different technologies may overlap in treatment, e.g. part of a UV light treatment may be performed in isolation and part may overlap with treatment(s) via ozone and/or hydrogen peroxide. For instance, nuts can be treated with a first treatment and a second treatment at the same time. Alternatively, nuts can be treated with a first treatment and a second treatment consecutively. In addition, one or both of the two technologies can be repeated, and/or an additional technology may be utilized as well. It is contemplated the combinations of treatment may allow for reduced dosage, strength, and/or duration of individual treatments, while achieving the same overall reduction of aflatoxin and/or the presence of Aspergillus flavus and/or Aspergillus parasiticus in an amount resulting in aflatoxin contamination It is also contemplated that combinations of treatment can provide further reduction of the amount of aflatoxin and/or the presence of Aspergillus flavus and/or Aspergillus parasiticus in an amount resulting in aflatoxin contamination. In certain embodiments, after the at least two treatments, the amount of aflatoxin is reduced by at least a half log, by at least one log, by at least 1.5 log, by at least 2 log, or at least 2.5 log.

In an embodiment of this aspect, nuts are treated with ultraviolet (UV) light and peroxide (e.g., hydrogen peroxide). In one embodiment, the UV light treatment and the hydrogen peroxide treatment are administered to the nuts sequentially. In one embodiment, the UV light treatment and the hydrogen peroxide treatment are administered to the nuts at substantially the same time. UV light is expected to accelerate production of radicals from hydrogen peroxide, which can improve reduction of aflatoxin. Treatment conditions can be adjusted as necessary. For instance, the amount of hydrogen peroxide can be from 0.1% H₂O₂ to 10% H₂O₂.

In an embodiment of this aspect, nuts are treated with ultraviolet (UV) light and ozone. In one embodiment, the UV light treatment and the ozone treatment are administered to the nuts sequentially. In one embodiment, the UV light treatment and the ozone treatment are administered to the nuts at substantially the same time. In one embodiment, the UV light treatment may partially overlap with the administration of one or more ozone treatments. In an embodiment, the UV light is from about 350 nm to about 365 nm, or about 140 nm to about 350 nm. In an embodiment, the UV light is from about 200 nm to 280 nm, such as 254 nm or 256 nm. In another embodiment, the UV light is less than about 200 nm, such as about 186 nm, or about 142 nm.

In an embodiment of this aspect, nuts are treated with ozone and peroxide. In one embodiment, the ozone treatment and the peroxide are administered to the nuts sequentially. In one embodiment, the ozone treatment and the peroxide treatment are administered to the nuts at substantially the same time. In one embodiment, the peroxide treatment may partially overlap with the administration of one or more ozone treatments. The ozone treatment may be gaseous ozone, aqueous ozone, or a combination thereof.

In an embodiment of this aspect, nuts are treated with ultraviolet (UV) light followed by supercritical carbon dioxide.

In an embodiment of this aspect, nuts are treated with pulsed electric field (PEF) followed by peroxide.

In an embodiment of this aspect, nuts are treated with peroxide followed by supercritical carbon dioxide.

Further combinations of treatments can be employed in the practice of the disclosed method. Moreover, combinations of three or more treatments are also contemplated in the practice of the disclosed method. The nuts can be treated with three or more different technologies at substantially the same time (i.e., in parallel), or can be treated with three or more treatments in series, or combinations thereof. For instance, nuts can be treated with a first treatment and a second treatment at the same time, followed by treatment with a third technology. In addition, one or more of the three different technologies can be repeated.

In an embodiment of this aspect, nuts are treated with ultraviolet (UV) light, ozone, and peroxide.

In an embodiment of this aspect, nuts are treated with ultraviolet (UV) light, ozone, and citric acid.

### Artificially-Contaminated Nuts

The disclosure further relates to a method of preparing aflatoxin- and/or Aspergillus-contaminated nuts. The method provides nuts having a specifiable contamination level, a reliable source of contaminated nuts, and permits in situ production. The contaminated nuts can be used, for instance, to test, develop, and/or optimize methods of decontamination in a more controlled manner, compared to naturally-contaminated nuts.

### Example 10: Aspergillus-Contaminated Nuts

Three strains of Aspergillus were identified for developing a method preparing aflatoxin-contaminated nuts, with a target of 200 ppb aflatoxin contamination. All three strains produce aflatoxin B1 and B2. The A. parasiticus strain also produces aflatoxin G1 and G2.

Each strain was tested for growth and aflatoxin production on clean nuts using two sets of incubation conditions and six timepoints. Temperatures tested were 25° C. and 30° C. and timepoints over 0-10 days were tested. Humidity was fixed at >97% relative humidity (RH). The nuts used were hazelnuts. The experimental protocol included (1) preparation of Aspergillus; (2) preparation of hazelnuts; (3) inoculation of hazelnuts; and (4) development of aflatoxin. These are described below.

### Preparation of Aspergillus

1. Prepare 10×90 mm Malt Extract Agar (MEA) plate cultures of Aspergillus flavus (ATCC #11498; CABI #52140), Aspergillus parasiticus (ATCC #16875; CABI #124931), and Aspergillus flavus (ATCC #22546; CABI #370082). Although CABI #124931 is designated as Aspergillus parasiticus in their catalog, ATCC #16875 is designated Aspergillus flavus. Catalog numbers for this Aspergillus flavus strain ATCC 16875 in different collections include: CBS 573.65; IFO 7540; QM 6738; WB 500; and NRRL 500.
2. Incubate plates at 25° C. for 10 days.
3. Examine microscopically to confirm formation of conidia.
4. In a biological safety cabinet, harvest conidia by swabbing and resuspend in 20 ml sterile deionized water with 0.1% Tween (SDWT).
5. Transfer conidial suspensions into centrifuge tubes and pellet conidia by centrifugation (4,000 rpm, 10 minutes).
6. Discard supernatant and resuspend conidial pellets in 20 mL SDWT.
7. Estimate conidial counts using a Neubauer counting chamber (WI-MB-11-025) and confirm by enumerating conidial suspension in triplicate on Aspergillus flavus and parasiticus agar (AFPA).
8. Adjust levels to 105 CFU/mL.
9. Store suspension at 2-8° C. until required (up to 1 week).

### Preparation of Nuts

1. Determine initial water activity and moisture levels of the hazelnuts.
2. Immerse samples in 1% sodium hypochlorite solution for 10 minutes to reduce levels of natural microflora/mycoflora.
3. Rinse with sterile distilled water to remove residual hypochlorite.
4. Remove most of residual water with absorbent paper.
5. For damaged hazelnuts, first place 1100 g of hazelnuts into a cement mixer and operate for 3 hours.

### Inoculation of nuts

1. Place 3×1,100 g hazelnut samples into large sterile stomacher bags.
2. Add ˜15 ml of A. parasiticus or A. flavus conidial suspension to each bag and mix thoroughly by hand to evenly distribute the inoculum over the sample.
3. Transfer into trays lined with filter paper and dry at ambient temperature under laminar flow of filtered air.
4. Check water activity periodically until it has returned to pre-inoculation levels.
5. Analyze triplicate samples to determine initial inoculum levels achieved after drying.

### Development of Aflatoxin

1. Transfer samples into sealed 50 L boxes containing 15 L sterile deionized water. Place nut samples onto a mesh tray in a single layer and suspend above the water at relative humidity >97%. Confirm humidity using a calibrated humidity meter.
2. Incubate samples at 30±2° C. for 4 days.
3. After incubation, take a small sample from each batch and image under UV light. Use uninoculated hazelnuts as a control image.
4. Take a sample of nuts immediately after incubation, wash (two conditions) and measure aflatoxin level to determine resilience of contamination.
   Wash condition 1: Water, 10 minutes
   Wash condition 2: 1% hypochlorite, 10 minutes, followed by water rinse
5. Spread out nuts in trays and dry them until their water activity/moisture has returned to pre-inoculation levels.
6. Take 5×50 g samples from each batch and analyze to determine aflatoxin levels using r-Biopharm® total aflatoxin ELISA kit (R-Biopharm AG, Darmstadt, Germany) with aflatoxin immunoaffinity columns.
7. From each batch, take triplicate samples on days 7, 14 and 21 and analyze to determine levels of aflatoxin using the r-Biopharm® total aflatoxin ELISA kit (R-Biopharm AG, Darmstadt, Germany).

Significant mold growth was observed on hazelnuts for all three strains tested and at both temperatures tested. Mold growth was significantly more extensive in the incubated samples than what is observed for natural nuts, which is attributed to the accelerated growth period. Mold plating showed that the mold growing on the nuts were all the inoculated Aspergillus, which is attributed to the washing of the nuts with sodium hypochlorite solution before inoculation; no sign of other mold species was seen.

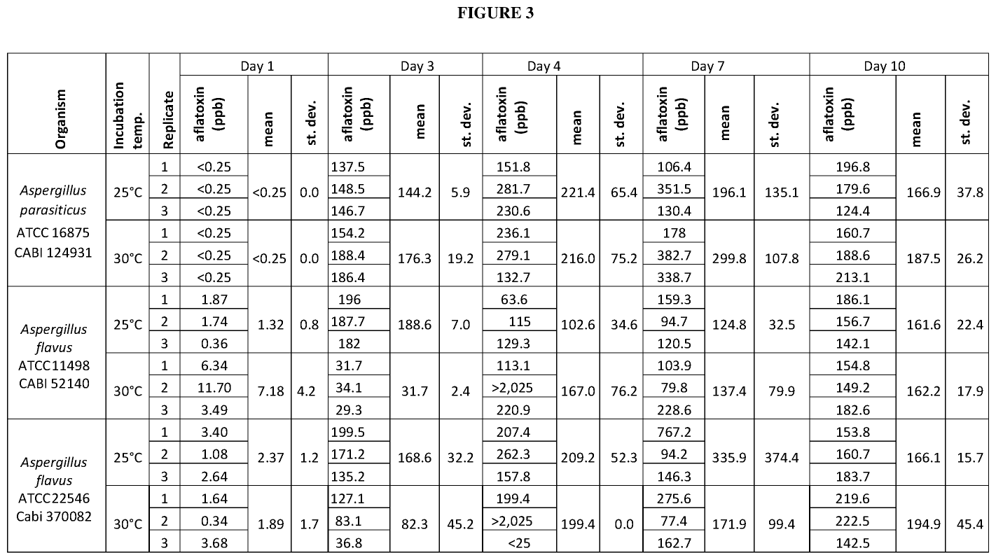

The data are shown in FIG. 3. There was high variability in aflatoxin levels. It is believed, without being held to theory, that the high variability was due to two factors. First, there was significant nut-to-nut variation in contamination due to different skin damage and corresponding mold growth levels. While this was high, the level observed here (˜1 order of magnitude) is far less than that in naturally-grown nuts (3-4 orders of magnitude). Second, Sample sizes for these measurements were small (10 g, ˜11 nuts). Testing on larger batches of nuts for technology screening is expected to reduce this uncertainty.

The results of the Aspergillus contamination trial showed several viable strains and incubation parameter sets that would be suitable to produce samples contaminated at the target 200 ppb level. Aspergillus flavus (ATCC #16875; CABI #124931) was chosen due to its consistent growth characteristics and tolerance to small variations in incubation times and temperatures, while still achieving the desired contamination level. The final contamination level was 216±75 ppb aflatoxin (30° C., Day 4), consistent with the target of 200 ppb.

### Example 11: Aflatoxin-Contaminated Nuts

Nuts were mixed with different volumes and concentrations of aflatoxin solution. Aflatoxin B1 solutions were prepared in methanol for the high volatility and solubility of aflatoxin. The aflatoxin levels and coating efficiencies measured.

The experimental protocol was as follows.
1. Prepare an aflatoxin B1 stock solution by dissolving 5 mg of aflatoxin B1 standard in 20 mL methanol (250 mg/L).
2. Transfer 1,336 μL (eq. 334 μg aflatoxin B1) into a separate 20 ml amber volumetric flask and fill to the line with methanol, and mix well by inverting. This gives 200 μg/kg (ppb), adjusted for the ˜60% transfer efficiency onto hazelnuts.
3. In a class I safety cabinet, carefully add the prepared solution to a sterile bag containing 1 kg hazelnuts to achieve a contamination level of 334 μg/kg (ppb) aflatoxin B1, ensuring that solution does not get onto the upper part of the bag.
4. Carefully heat seal the bag, ensuring a proper seal is achieved.
5. Mix thoroughly by hand for 2-3 minutes to evenly distribute toxin across hazelnuts.
6. Leave samples in the safety cabinet for aerosols to completely settle.
7. In safety cabinet, pour contaminated hazelnuts into trays lined with filter paper to achieve an even layer.
8. Dry overnight within a class 1 cabinet at ambient temperature with air flow to allow complete evaporation of methanol, leaving aflatoxin on nut surfaces.
9. Transfer batch to a large plastic bag and mix gently but thoroughly.
10. Extract 10×75 g samples selected randomly from throughout each batch and measure aflatoxin concentration by chosen method (ELISA for Aspergillus mold inoculation samples and HPLC for direct aflatoxin contamination samples) to determine success, degree, and uniformity of contamination.

The data are shown in Table 2. Overall contamination levels were 216±25 ppb (sample size of 30 g), consistent with the target of 200 ppb. A coating efficiency of around 60% was achieved, as apparently some aflatoxin was lost to the inside of the mixing bags and drying trays.

TABLE 2

| Replicate | A | B | C | Inner Batch |
|---|---|---|---|---|
| 1 | 202 | 211 | 273 | |
| 2 | 259 | 199 | 222 | |
| 3 | 243 | 205 | 204 | |
| 4 | 262 | 205 | 187 | |
| 5 | 219 | 224 | 197 | |
| 6 | 252 | 224 | 184 | |
| 7 | 261 | 231 | 211 | |
| 8 | 251 | 230 | 200 | |
| 9 | 217 | 186 | 197 | |
| 10 | 181 | 209 | 192 | |
| Mean | 235 | 216 | 195 | 216 |
| Standard | 15 | 26 | 15 | 25 |
| Deviation | | | | |

The invention is further described in detail by reference to the above experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The disclosures of each and every patent, patent application, and publication cited herein are hereby incorporated herein by reference in their entirety.

Although the present embodiments have been described in detail with reference to examples above, it is understood that various modifications can be made without departing from the spirit of these embodiments, and would readily be known to the skilled artisan. The appended claims are intended to be construed to include all such embodiments and equivalent variations.

What is claimed is:

1. A method of reducing aflatoxin contamination in one or more nuts and/or reducing the amount of Aspergillus flavus and/or Aspergillus parasiticus resulting in aflatoxin contamination on one or more nuts, comprising contacting one or more nuts with one or more treatments, thereby reducing the presence of aflatoxin and/or reducing the amount of Aspergillus flavus and/or Aspergillus parasiticus resulting in the presence of aflatoxin, relative to the one or more nuts prior to contact with the one or more treatments.

2. The method of claim 1, wherein the one or more treatments is selected from ultraviolet (UV) light, ozone (O₃), peroxide, electrolyzed oxidizing water (EOW), pulsed electric field (PEF), supercritical carbon dioxide, cold plasma, organic acid wash, and combinations thereof.

3. The method of claim 1, wherein the one or more nuts is contacted with two treatments selected from ultraviolet (UV) light, ozone (O₃), peroxide, electrolyzed oxidizing water (EOW), pulsed electric field (PEF), supercritical carbon dioxide, cold plasma, organic acid wash, and combinations thereof.

4. The method of claim 3, wherein the one or more nuts is contacted with two treatments selected from ultraviolet (UV) light, ozone (O₃), and peroxide.

5. The method of claim 3, wherein the one or more nuts is contacted with ultraviolet (UV) light and ozone (O₃), or ultraviolet (UV) light and peroxide.

6. The method of claim 3, wherein the one or more nuts is contacted with ozone (O₃) and peroxide.

7. The method of claim 2, wherein the one or more nuts is contacted with ultraviolet (UV) light, ozone (O₃), and peroxide.

8. The method of claim 1, wherein the one or more nuts is contacted with three treatments selected from ultraviolet (UV) light, electrolyzed oxidizing water (EOW), ozone (O₃), peroxide, pulsed electric field (PEF), supercritical carbon dioxide, cold plasma, organic acid wash, and combinations thereof.

9. The method of claim 8, wherein the one or more nuts is contacted with ultraviolet (UV) light, ozone (O₃), and peroxide.

10. The method of claim 1, wherein the nuts are almonds, cashews, hazelnuts, macadamias, peanuts, pecans, pistachios, walnuts, or combinations thereof.
